(19) United States
(12) Patent Application Publication
(10) Pub. No.: US 2026/0102577 A1
(43) Pub. Date: Apr. 16, 2026

(54) SYSTEMS AND METHODS FOR CARDIOGENIC OSCILLATION DETECTION

(71) Applicant: RESMED SENSOR TECHNOLOGIES LIMITED, Dublin (IE)

(72) Inventors: Anna RICE, Dublin (IE); Hannah Meriel KILROY, Dublin (IE); Graeme Alexander LYON, Dublin (IE)

(21) Appl. No.: 19/116,792

(22) PCT Filed: Sep. 27, 2023
(86) PCT No.: PCT/IB2023/059652

§ 371 Date: Mar. 28, 2025

Related U.S. Application Data
(60) Provisional application No. 63/411,383, filed on Sep. 29, 2022.

(51) Int. Cl. A61M 16/00

(52) U.S. Cl. CPC A61M 16/024; A61M 16/0066; A61M 2016/0033; A61M 2230/06

(57) ABSTRACT

A method includes receiving a flow rate signal associated with a user interface of a respiratory therapy device used by a user engaging in a sleep session. The method further includes identifying a plurality of cardiogenic oscillations from the flow rate signal. The method further includes determining consecutive peak distances based at least in part on the identified plurality of cardiogenic oscillations. The method further includes calculating heart rate information based on the consecutive peak distances.

### CROSS-REFERENCE TO RELATED APPLICATIONS

This application claims the benefit of, and priority to, U.S. Provisional Patent Application No. 63/411,383, filed Sep. 29, 2022, which is hereby incorporated by reference herein in its entirety.

### TECHNICAL FIELD

The present disclosure relates generally to systems and methods for identifying heart rate information, and more particularly, to systems and methods for identifying heart rate information from a respiratory therapy device.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. Generally, pressurized air is monitored to facilitate operation of the respiratory therapy system. Leveraging these monitored signals for other uses, however, can be difficult. US 2015/182713 describes that the presence of cardiogenic pressure or flow oscillations can be sensed and used to adjust triggering of a pressure apparatus. However, detection of the presence of these cardiogenic oscillations provides limited information. Determining specific heart rate information, especially to the extent necessary for such heart rate information to be leveraged for certain uses, is problematic, especially due to the nature of how such oscillations appear in a flow or pressure signal. The present disclosure is directed to solving these and other problems.

### SUMMARY

According to some implementations of the present disclosure, a method includes receiving a flow rate signal associated with air supplied to airways of a user engaging in a sleep session. The air is supplied by a respiratory therapy device. The method further includes identifying a plurality of cardiogenic oscillations from the flow rate signal. The method further includes determining consecutive peak distances based at least in part on the identified plurality of cardiogenic oscillations. The method further includes calculating heart rate information based on the consecutive peak distances.

According to some implementations of the present disclosure, a system includes a flow generator of a respiratory therapy device for supplying pressurized air to airways of a user engaging in a sleep session. The system further includes a flow rate sensor for supplying a flow rate signal associated with the supplied pressurized air. The system further includes a control system comprising one or more processors. The system further includes a non-transitory computer readable medium having thereon machine executable instruction, which, when executed by the one or more processors, cause the control system to perform operations including receiving the flow rate signal. The operations further include identifying a plurality of cardiogenic oscillations from the flow rate signal. The operations further include determining consecutive peak distances based at least in part on the identified plurality of cardiogenic oscillations. Detecting the plurality of cardiogenic oscillations includes detecting local peaks in the flow rate signal that satisfy a plurality of thresholds for a plurality of features. The plurality of features includes peak prominence, peak amplitude, and peak width. The operations further include calculating heart rate information based on the consecutive peak distances.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 illustrates an exemplary timeline for a sleep session, according to some implementations of the present disclosure.

FIG. 4 illustrates an exemplary hypnogram associated with the sleep session of FIG. 3, according to some implementations of the present disclosure.

FIG. 5 is a chart depicting flow rate over time showing cardiogenic oscillations according to certain aspects of the present disclosure.

FIG. 6 is a chart depicting flow rate over time showing primary and secondary cardiogenic oscillations according to certain aspects of the present disclosure.

FIG. 7 is a set of histograms depicting the frequency of heart rate and peak width across a flow rate signal during an example sleep session where the cardiogenic oscillations can be differentiated into multiple clusters of peak feature values, according to certain aspects of the present disclosure.

FIG. 8 is a set of histograms depicting the frequency of heart rate and peak width across a flow rate signal during an example sleep session, where the cardiogenic oscillations follow morphology single cluster of peak feature values, according to certain aspects of the present disclosure.

FIG. 9 is a flowchart depicting a process for identifying cardiogenic oscillations and determining heart rate information according to certain aspects of the present disclosure.

FIG. 10 is a combination chart depicting cardiogenic oscillations present in pressure and flow signals over time, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Certain aspects and features of the present disclosure relate to techniques for obtaining and leveraging heart rate information from airflow data (e.g., a flow signal, such as a flow rate signal and/or a flow pressure signal) of a respiratory therapy device. The flow signal can be processed to identify cardiogenic oscillations using techniques for improved peak detection and artifact removal. Cardiogenic peaks can be identified based on one or more peak features falling within trained threshold value(s). Artifact removal can include applying filters to the heart rate information and/or histogram(s) of one or more peak features to identify a portion of the heart rate information to retain and/or a portion of the heart rate information to exclude. The resultant heart rate information can be leveraged to improve analytics associated with use of the respiratory therapy device and/or the user's overall sleep therapy; can be leveraged to provide cardiac-specific coaching and information to a user; can be leveraged to augment remote patient care data; and can be leveraged for other uses.

Cardiogenic oscillations (CGOs) are small, periodic oscillations, found in a signal, that relate to beating of the heart. More specifically, CGOs are often present in the flow rate signals and pressure signals associated with a respiratory therapy system in use. In flow rate signals and flow pressure signals, CGOs are most often seen during central apneas and, at least in some cases, during expiration. Identification of CGO peaks, however, is difficult due to many factors, such as inconsistency of the presence of CGOs in a flow rate or flow pressure signal, and the similarity of CGO peaks with other nearby peaks in the flow or pressure signal.

In some cases, it can be advantageous to identify CGOs from flow rate data (e.g., flow rate signals), especially because of the ease of obtaining and accessing flow rate signals from a user of a respiratory therapy device. Identifying CGO peaks in flow rate signals, however, can be challenging. In flow rate signals, CGO peaks are most often seen during exhalation, but not necessarily during every exhalation. In some instances, CGO presents as a primary and secondary peak. In such cases, differentiating the primary CGO peak from secondary CGO peaks can be difficult. Further, flow rate signals are obtained at sampling rates at or around 25 Hz, which while useful for measurement of respiration information, is not necessarily useful for measuring heart rate information. For example, if a CGO peak would naturally fall somewhere in time between two samples, the flow rate signal would not accurately show the location of the CGO peak in time, rather forcing the peak to the previous or subsequent sample. As a result, accurate heart rate information can be difficult to obtain, especially depending on the type of heart rate information (e.g., higher accuracy of heart rate measurements may be important when attempting to measure and leverage heart rate variability data). While increasing the sampling rate is an option to address this problem, doing so is not always advantageous as it increases the power and memory requirements of the underlying system, as well as overall cost. Thus, it can be important to find techniques for accurately identifying CGO peaks in flow rate signals.

While described herein with reference to flow rate signals, the disclosed aspects and features of the present disclosure can be used to identify CGO peaks and determine CGO-derived heart rate information from any flow signal, such as a flow pressure signal (also known in the art as a “pressure signal”). In such signals, CGO peaks and CGO-derived heart rate information may be derived via methods analogous to those described herein in respect of flow rate signals. Similarly, CGO peaks and determined CGO-derived heart rate information may be derived from a signal generated by an acoustic sensor, such as a microphone. As may be understood, an acoustic sensor such as a microphone is a form of pressure sensor which measures sound pressure variations and converts to an electrical signal. In embodiments, the acoustic sensor, e.g., microphone, may be located within, or otherwise physically integrated with, a respiratory therapy system and in acoustic communication with the flow of air which, in operation, is generated by the flow generator of the respiratory therapy device comprised in the respiratory therapy system. Processing of the acoustic data generated by the acoustic sensor may be employed to remove undesired components from the audio signal comprised in the acoustic data. This may include filtering of the acoustic data to retain only the frequencies associated with the cardiac cycle, for example a band-pass filter in the range of 0.5-3.5 Hz. Other components which may be comprised in the audio signal, such as physiological signals attributable to breathing or known device-generated signals (e.g., motor RPM, air flow signals used in forced oscillation technique (FOT)), and these may similarly be removed through filtering or other signal decomposition techniques as described in the art.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal (e.g., flow rate signal) derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

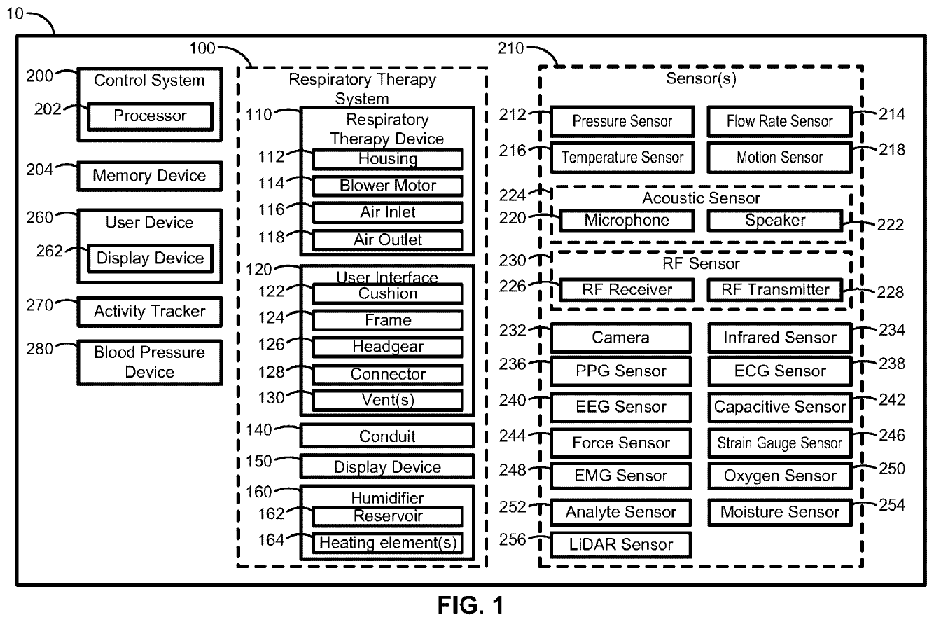

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

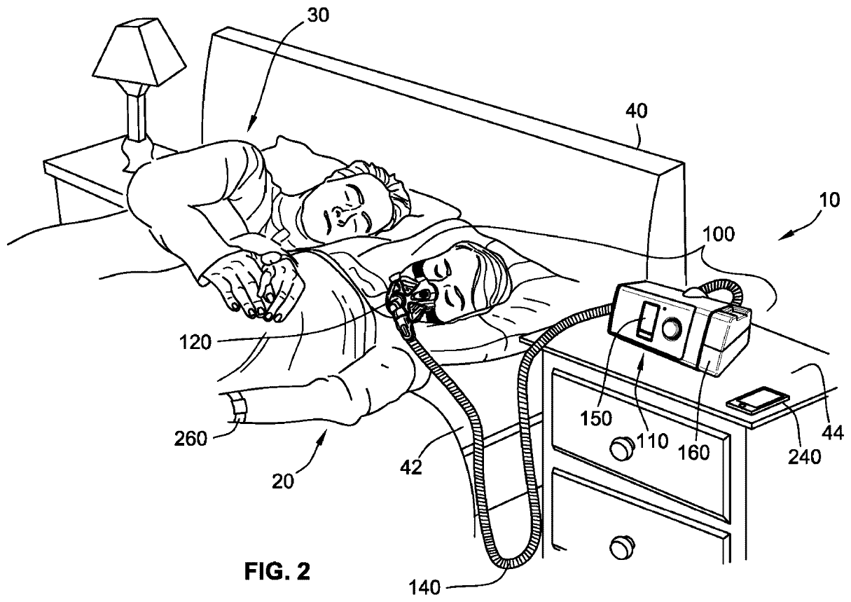

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about-20 L/min and about 150 L/min, while maintaining a positive pressure (relative to the ambient pressure).

Referring back to FIG. 1, the respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). In some cases, the housing 112 can include a vent 113 to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

In some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

In some cases, the cushion 122 and frame 124 of the user interface 120 form a unitary component of the user interface 120. The user interface 120 can also include a headgear 126, which generally includes a strap assembly and optionally a connector 128. The headgear 126 can be configured to be positioned generally about at least a portion of a user's head when the user wears the user interface 120. The headgear 126 can be coupled to the frame 124 and positioned on the user's head such that the user's head is positioned between the headgear 126 and the frame 124. The cushion 122 can be positioned between the user's face and the frame 124 to form a seal on the user's face. The optional connector 128 can be configured to couple to the frame 124 and/or cushion 122 at one end and to a conduit 140 of a respiratory therapy system 100. The pressurized air can flow directly from the conduit 140 of the respiratory therapy system 100 into the volume of space defined by the cushion 122 (or cushion 122 and frame 124) of the user interface 120 through the connector 128. From the user interface 120, the pressurized air reaches the user's airway through the user's mouth, nose, or both. Alternatively, where the user interface 120 does not include the connector 128, the conduit of the respiratory therapy system can connect directly to the cushion 122 and/or the frame 124.

In some implementations, the connector 128 may include one or more vents 130 (e.g., a plurality of vents) located on the main body of the connector 128 itself and/or one or a plurality of vents 130 (“diffuser vents”) in proximity to the frame 124, for permitting the escape of carbon dioxide (CO₂) and other gases exhaled by the user. In some implementations, one or a plurality of vents 130 may be located in the user interface 120, such as in frame 124, and/or in the conduit 140. In some implementations, the frame 124 includes at least one anti-asphyxia valve (AAV), which allows CO₂ and other gases exhaled by the user to escape in the event that the vents 130 fail when the respiratory therapy device is active. In general, AAVs are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 130, other masks might have only the plurality of vents 130 on the connector 128 itself).

In some cases, the user interface 120 can be an indirect user interface. Such an interface 120 can include a headgear 126 (e.g., as a strap assembly), a cushion 122, a frame 124, a connector 128, and a user interface conduit (often referred to as a minitube or a flexitube). The user interface 120 is an indirectly connected user interface because pressurized air is delivered from the conduit 140 of the respiratory therapy system to the cushion 122 and/or frame 124 through the user interface conduit, rather than directly from the conduit 140 of the respiratory therapy system.

In some implementations, the cushion 122 and frame 124 form a unitary component of the user interface 120. Generally, the user interface conduit is more flexible than the conduit 140 of the respiratory therapy system 100 described above and/or has a diameter smaller than the diameter of the than the than the conduit 140. The user interface conduit is typically shorter that conduit 140. The headgear 126 of such a user interface 120 can be configured to be positioned generally about at least a portion of a user's head when the user wears the user interface 120. The headgear 126 can be coupled to the frame 124 and positioned on the user's head such that the user's head is positioned between the headgear 126 and the frame 124. The cushion 122 is positioned between the user's face and the frame 124 to form a seal on the user's face. The connector 128 is configured to couple to the frame 124 and/or cushion 122 at one end and to the conduit of the user interface 120 at the other end. In other implementations, the user interface conduit may connect directly to frame 124 and/or cushion 122. The user interface conduit, at the opposite end relative to the frame 124 and cushion 122, is configured to connect to the conduit 140. The pressurized air can flow from the conduit 140 of the respiratory therapy system, through the user interface conduit, and the connector 128, and into a volume of space define by the cushion 122 (or cushion 122 and frame 124) of the user interface 120 against a user's face. From the volume of space, the pressurized air reaches the user's airway through the user's mouth, nose, or both.

In some implementations, the connector 128 includes a plurality of vents 130 for permitting the escape of carbon dioxide (CO₂) and other gases exhaled by the user when the respiratory therapy device is active. In such implementations, each of the plurality of vents 130 is an opening that may be angled relative to the thickness of the connector wall through which the opening is formed. The angled openings can reduce noise of the CO₂ and other gases escaping to the atmosphere. Because of the reduced noise, acoustic signal associated with the plurality of vents 130 may be more apparent to an internal microphone, as opposed to an external microphone. Thus, an internal microphone may be located within, or otherwise physically integrated with, the respiratory therapy system and in acoustic communication with the flow of air which, in operation, is generated by the flow generator of the respiratory therapy device, and passes through the conduit and to the user interface 120.

In some implementations, the connector 128 optionally includes at least one valve 130 for permitting the escape of CO₂ and other gases exhaled by the user when the respiratory therapy device is inactive. In some implementations, the valve 130 (an example of an anti-asphyxia valve) includes a silicone (or other suitable material) flap that is a failsafe component, which allows CO₂ and other gases exhaled by the user to escape in the event that the vents 130 fail when the respiratory therapy device is active. In such implementations, when the silicone flap is open, the valve opening is much greater than each vent opening, and therefore less likely to be blocked by occlusion materials.

In some cases, the user interface 120 can be an indirect headgear user interface 120 and can include headgear 126, a cushion 122, and a connector 128. The headgear 126 includes strap and a headgear conduit. The headgear 126 is configured to be positioned generally about at least a portion of a user's head when the user wears the user interface 120. The headgear 126 includes a strap that can be coupled to the headgear conduit and positioned on the user's head such that the user's head is positioned between the strap and the headgear conduit. The cushion 122 is positioned between the user's face and the headgear conduit to form a seal on the user's face.

In such cases, the connector 128 can be configured to couple to the headgear 126 at one end and a conduit 140 of the respiratory therapy system 100 at the other end. In other implementations, the connector 128 is not included and the headgear 126 can alternatively connect directly to conduit 140 of the respiratory therapy system 100. The headgear conduit can be configured to deliver pressurized air from the conduit 140 of the respiratory therapy system 100 to the cushion 122, or more specifically, to the volume of space around the mouth and/or nose of the user and enclosed by the user cushion 122. The headgear conduit is hollow to provide a passageway for the pressurized air. Both sides of the headgear conduit can be hollow to provide two passageways for the pressurized air. Alternatively, only one side of the headgear conduit can be hollow to provide a single passageway. In some cases, headgear conduit comprises two passageways which, in use, are positioned at either side of a user's head/face. Alternatively, only one passageway of the headgear conduit can be hollow to provide a single passageway. The pressurized air can flow from the conduit 140 of the respiratory therapy system 100, through the connector 128 and the headgear conduit, and into the volume of space between the cushion 122 and the user's face. From the volume of space between the cushion 122 and the user's face, the pressurized air reaches the user's airway through the user's mouth, nose, or both.

In some implementations, the cushion 122 includes a plurality of vents 130 on the cushion 122 itself. Additionally, or alternatively, in some implementations, the connector 128 includes a plurality of vents 130 (“diffuser vents”) in proximity to the headgear 126, for permitting the escape of carbon dioxide (CO₂) and other gases exhaled by the user when the respiratory therapy device is active. In some implementations, the headgear 126 may include at least one plus anti-asphyxia valve (AAV) in proximity to the cushion 122, which allows CO₂ and other gases exhaled by the user to escape in the event that the vents 130 fail when the respiratory therapy device is active.

The conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 can include a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. In an example, air can flow from an air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260. In implementations, the processor 202 and/or memory device 204 may be comprised in a user device 260, which may be an electronic device such as a smartphone, tablet, or other computer device. Data, such as a flow signal or data generated therefrom, may be received at the user device, such as via a wired or wireless connection with a respiratory therapy device. Such data may be stored in memory device 204 of the user device and/or analyzed by the processor 202 of the user device to identify cardiogenic oscillations and/or other physiological parameters, determine consecutive peak distances based at least in part on the identified plurality of cardiogenic oscillations, and/or calculate heart rate information based on the consecutive peak distances, as described herein. Other data may also be received at the user device 260, such as physiological data and/or audio data, from the one or more sensors 210.

The one or more sensors 210 include a flow sensor such as pressure sensor 212 and/or flow rate sensor 214, a temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data (e.g., a flow pressure signal) that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data (e.g., a flow rate signal) that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum heart respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

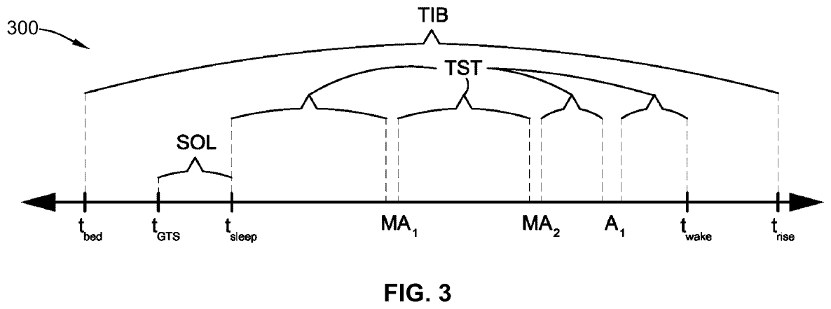

Referring to the timeline 300 in FIG. 3 the enter bed time t_{bed} is associated with the time that the user initially enters the bed (e.g., bed 40 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (tors) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

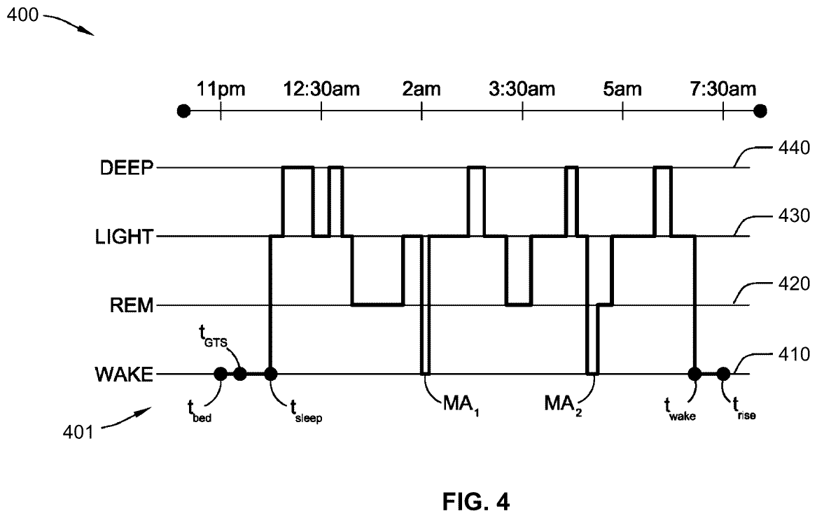

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (tors) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 3), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 3), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (tors), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (tors), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

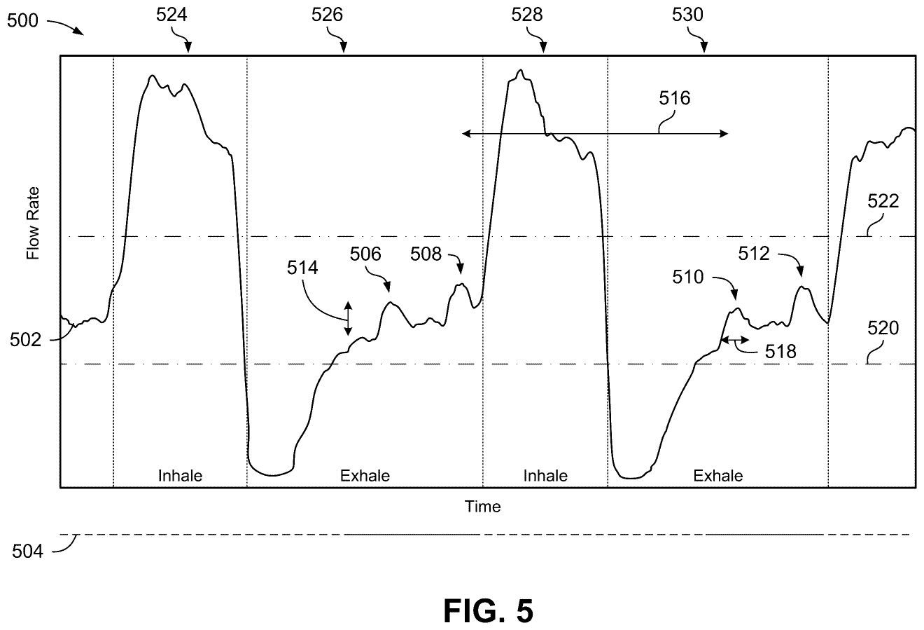

FIG. 5 is a chart 500 depicting flow rate over time showing cardiogenic oscillations according to certain aspects of the present disclosure. The flow rate is shown by a flow signal 502, which traces the flow rate over time of pressurized air from a respiratory therapy device through a conduit and/or user interface (e.g., user interface 120 of FIG. 1) to a user's airways. The flow signal 502 can be measured by one or more sensors (e.g., flow rate sensor(s) 214 of FIG. 1).

The flow signal 502 can be a flow signal that has been filtered according to one or more filters, such as i) a smoothing filter or low-pass filter to remove noise; ii) a Gaussian or Laplacian filter to exaggerate CGO peaks; iii) a respiratory-therapy-device filter that filters out diagnostic flow signals introduced by the respiratory therapy device itself, such as forced oscillation technique (FOT) signals (e.g., a pulse of pressure at or around 4 Hz to facilitate identification and/or discrimination of obstructive and central sleep apnea), which can be a notch filter (e.g., a 4 Hz notch filter); or iv) any combination of i-iii.

The flow signal 502 shows a user inhaling and exhaling while using a respiratory therapy device (e.g., respiratory therapy device 100 of FIG. 1). Chart 500 depicts a series of inhalation and exhalation segments 524, 526, 528, 530. The system can identify the boundaries of segments 524, 526, 528, 530 automatically based on flow direction, although other techniques may be used. As seen in chart 500, CGO peaks 506, 508, 510, 512 are visible in exhalation segments 526, 530. In some cases, CGO peaks may be additionally present in inhalation segments 524, 528, although it has been determined that the CGO peaks present in exhalation segments 526, 530 are more reliably identified. In some cases, CGO peaks are absent from exhalation segments 526, 530 despite the user's heart beating at the time.

Identification of CGO peaks 506, 508, 510, 512 can be based primarily on detection of local peaks in the flow signal 502. Any given local peak may be identified as being a CGO peak based on various peak features. Peak prominence 514 is an indication of a peak's amplitude above local flow rate levels (e.g., amplitude above a baseline flow rate if no peak were present). To qualify as a CGO peak, the peak may need to have a peak prominence 514 at least greater than a threshold value. Peak width 518 is an indication of the duration of time of the peak (e.g., time between a peak starting time and a peak ending time). To qualify as a CGO peak, the peak may need to have a peak width 518 that is at least greater than a threshold value. A peak amplitude can be a value (e.g., flow rate value) of the tip of the peak (e.g., from zero to the tip of the peak). To qualify as a CGO peak, the peak may need to have an amplitude that exceeds a lower amplitude threshold 520 and/or does not exceed an upper amplitude threshold 522. A peak distance 516 is a duration of time between peaks. To qualify as a CGO peak, the peak distance 516 may need to be within a threshold range or at least below a threshold value. In some cases, identification of CGO peaks 506, 508, 510, 512 can make use of some or all of these features, and/or other features of the flow rate signal 502.

Threshold values and/or ranges of features used to identify CGO peaks, 506, 508, 510, 512 can be trained based on training data that includes a flow signal 502 coordinated with a reference signal. The reference signal can be an oximetry signal, an ECG signal, or other signal associated with heart rate. The reference signal can be used to identify heart beats (e.g., as identified by QRS complexes of an ECG signal), which can then be correlated to points on the flow rate signal 502. The training system can then automatically adjust threshold values and/or ranges of features used to identify CGO peaks such that the peaks identified as CGOs correlate with the heart beats identified in the reference signal. In some cases, feature thresholds (e.g., threshold values and/or threshold ranges) can be trained for a corpus of individuals and used for future individuals. In some cases, user-specific feature thresholds can be trained for an individual based on training data acquired for that individual. In some cases, the degree of fit of the training can be adjusted depending on a desired balance between CGO peak identification accuracy and information loss.

As the degree of fit increases, the identified peaks are more likely to be correctly identified CGO peaks (e.g., increased identification accuracy), but some CGO peaks may be not identified, thus resulting in information loss. In some cases, different feature thresholds can be used for different purposes. For example, for use cases where a smaller number of more accurate heart rate information data points is needed, a first set of features thresholds can be used. However, for use cases where a larger number of heart rate information data points are needed and less accuracy is acceptable, a second set of features thresholds can be used

Eventually, after CGO peaks 506, 508, 510, 512 are identified, the distances between consecutive peaks can be calculated and used to calculate a heart rate. In some cases, if the peak distance 516 is greater than a threshold value, an assumption can be made that a CGO peak may be missing or otherwise not identified, in which case the heart rate calculation can ignore that distance or apply logic to calculate heart rate (e.g., assume the presence of one or more CGO peaks within that distance. In some cases, distances between consecutive CGO peaks 506, 508,510, 512 are used to calculate heart rate only for consecutive CGO peaks within the same segment 524, 526, 528, 530. For example, CGO peak 560 and CGO peak 508 both fall within exhalation segment 526, and thus the distance between them can be used to calculate a heart rate. However, CGO peak 508 and CGO peak 510 are not in the same segment, and are in fact separated by segment 528, and thus the distance between them can be ignored when calculating heart rate.

Distance between consecutive peaks can be represented as a sample distance (d) or a time interval (t). The sample distance (d) between consecutive peaks can be calculated as a number of samples between the location of a first peak and the location of a second peak, such as in the equation d=loc₂−loc₁, where loc₁ and loc₂ refer to the sample indices of the first and second peaks, respectively. The time interval (t) between consecutive peaks can be calculated as the sample distance divided by the sampling frequency (fₛ) of the signal, such as in the equation t=d/fₛ. Once a distance between consecutive peaks is obtained, heart rate in beats per minute can be calculated as 60 divided by the time interval, such as HR=60/t, where HR is heart rate in beats per minute. In another example, heart rate in beats per minute can be calculated according to the equation HR=60/((loc_2−loc_1)/f_s).

The presence and absence of CGO peaks 506, 508, 510, 512 can be represented by a CGO presence signal 504. The CGO presence signal 504 is an indication of CGO uptime, or the amount of time that CGO peaks are present in a flow rate signal 502. CGO uptime can be broken down by any suitable units of time and can be represented as a number of that unit of time during which CGO peaks are present in the flow signal. For example, CGO uptime can be broken down by minutes, such that CGO uptime is the count of each minute of the flow rate signal 502 where a CGO peak is identified (e.g., CGO peaks were present for 100 minutes out of a 360-minute flow rate signal). CGO uptime can also be represented as a percentage (e.g., CGO peaks present for 100 minutes of a 360-minute flow rate signal may correspond to a CGO uptime of approximately 27.8%).

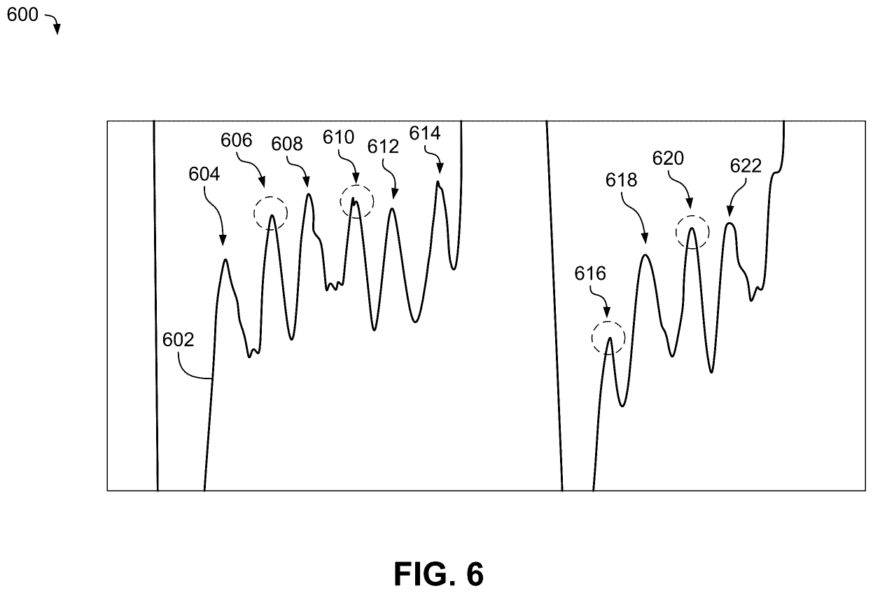

FIG. 6 is a chart 600 depicting flow rate over time showing primary and secondary cardiogenic oscillations according to certain aspects of the present disclosure. Flow rate signal 602 is any suitable flow rate signal (e.g., flow rate signal 502 of FIG. 5) associated with airflow generated by a respiratory therapy device and directed into a user's airways. In some cases, however, flow rate signal 602 can include multiple local peaks that may each appear to be individual CGO peaks, although they relate to the same underlying heartbeat. Put another way, a single heartbeat can generate a primary cardiogenic oscillation and, in some cases, one or more secondary cardiogenic oscillations. Thus, when calculating heart rate information from cardiogenic oscillations, a true heart rate would be calculated when considering only primary cardiogenic oscillations, and a heart rate calculated using primary and secondary cardiogenic oscillations may be artificially high (e.g., doubled).

The flow rate signal 602 shows such an example signal from two adjacent breaths. Flow rate signal 602 includes many localized peaks 604, 606, 608, 610, 612, 614, 616, 618, 620, 622, each of which has been identified as a cardiogenic oscillation, across these two breaths. However, not all of these peaks are primary CGO peaks. If the system were to merely identify a heart rate from all of these peaks as CGO peaks, an artificially high heart rate would be calculated. Thus, can be important to properly differentiate primary CGO peaks from secondary CGO peaks, or at least determine when secondary CGO peaks exist in the flow rate signal 602.

In some cases, primary CGO peaks can be differentiated from secondary CGO peaks, such as by comparing peak feature values of the peaks, thus allowing the system to calculate heart rate information based on only the primary CGO peaks (or only the secondary CGO peaks, when a single secondary CGO peak exists for each primary CGO peak). Certain aspects of the present disclosure differentiate CGO peaks 606, 610, 616, 620 from secondary peaks 604, 608, 612, 614, 618, 622 based on morphology of the peaks. The peaks that have peak feature values similar to those of other CGO peaks can be considered to be CGO peaks. One or more peak feature (e.g., peak width) can be used. In some cases, primary and secondary CGO peaks may appear similar, but may have different peak widths. For example, in some cases, the primary CGO peaks are narrower than the secondary CGO peaks. Differentiation of CGO peaks and secondary peaks is disclosed in further detail herein.

In some cases, however, a determination can be made that a flow rate signal 602 includes both primary and secondary CGO peaks. In response to this determination, optionally without determining which CGO peaks are primary or secondary, calculated heart rate information can be adjusted, ignored, or otherwise processed to produce an accurate heart rate. For example, if multiple clusters of peak feature values are detected across the identified CGO peaks, it can be determined that secondary CGO peaks exist, and heart rate estimates that appears to be doubled can be ignored, while heart rate estimates that are half of the suspected-doubled estimates can be used.

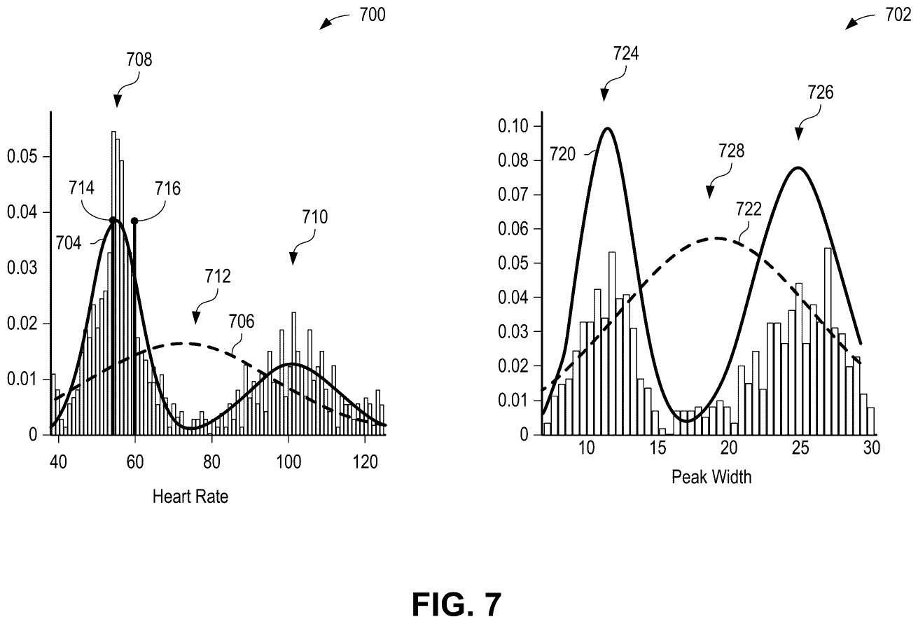

FIG. 7 is a set of histograms 700, 702 depicting the frequency of calculated heart rate estimates and CGO peak widths across a flow rate signal during an example sleep session where the cardiogenic oscillations can be differentiated into multiple clusters by peak feature values, according to certain aspects of the present disclosure. The flow rate signal can be a flow rate signal similar to flow rate signal 502 of FIG. 5 or flow rate signal 602 of FIG. 6.

Histogram 700 depicts a collection of heart rate estimates calculated from the various CGOs identified in the flow rate signal. More specifically, the histogram 700 depicts the frequency with which each of the depicted heart rates was estimated from the identified CGO peaks. As depicted in histogram 700, more heart rate estimates were identified around 55-59 beats per minute than at other heart rates.

By applying Gaussian mixture modeling, or a similar cluster modeling approach, one or more curves 704, 706 can be generated to identify clusters of heart rate estimates. The modeling approach (e.g., Gaussian mixture modeling) can be configured to try and find a certain number of cluster peaks corresponding to that same number of heart rate estimates clusters. For example, if it is presumed that a single heart rate cluster exists in the flow rate signal, the modeling approach can be configured to identify a single cluster, which would result in generation of curve 706, which identifies a single curve peak 712 at around 78 beats per minute.

Alternatively, if it is presumed that two heart rate clusters exist in the flow rate signal, the modeling approach can be configured to identify two clusters, which would result in generation of curve 704, which identifies a first curve peak 708 at around 57 beats per minute and a second curve peak 710 at around 101 beats per minute. Other numbers of heart rate clusters, such as three or more, can be sought by configuring the appropriate modeling approach.

In some cases, a preset number of potential heart rate clusters can be used (e.g., two, three, or more) in the generation of the curve of heart rate clusters.

In some cases, however, the number of potential heart rate clusters to be used can be obtained through analysis of the flow rate signal. In an example, the number of potential heart rate clusters to be used to configure the modeling approach can be based on the number of identified clusters of peak feature values in the collection of identified CGO peaks.

In some cases, even when multiple peaks are sought, the number of identified clusters of peak feature values in the collection of identified CGO peaks can be used to determine which of the multiple peaks to select.

Histogram 702 depicts a collection of peak width values measured from the various CGO peaks identified in the flow rate signal. More specifically, histogram 702 depicts the frequency with which each of the depicted peak width values were measured in the various identified CGOs from the flow rate signal. Similarly to as described above, a modeling approach (e.g., Gaussian mixture modeling) can be used to identify cluster peaks in the histogram, each of which is indicative of a discernable cluster of peak feature values. While histogram 702 depicts peak width, it will be understood that clusters of peak feature values for any combination of one or more peak features, such as those peak features discussed with reference to FIG. 5, can be used to differentiate CGO peaks.

The number of peaks sought using the modeling approach can be preset (e.g., one, two, three, or more), although that need not always be the case. In an example of a single peak being sought, curve 722 depicts a single curve peak 728, representing a single cluster of peak feature values for CGO peaks having a peak width of approximately 17 units (any appropriate unit of measurement of the peak feature can be used). Since the frequency of peak widths at that value is relatively low compared to other, it can be presumed that curve 722 does not provide an accurate fit. However, when two peaks are sought, curve 720 can be generated, which depicts a first curve peak 724 and a second curve peak 726, with peak width values of approximately 13 and 25, respectively. The frequencies of peak widths at those peak width values are relatively high compared to others, so it may be presumed that curve 720 provides an accurate fit.

The number of clusters of peak feature values (e.g., number of peaks in a well-fitting curve, such as two peaks from curve 720), identified from the chosen peak feature(s) can be used to set the number of peaks sought in the modeling approach used to generate a curve in the heart rate histogram 700.

Thus, as depicted in FIG. 7, since two peaks were identified in the peak feature histogram 702, a modeling approach seeking two peaks was used in the heart rate histogram 700, identifying first peak 708 and second peak 710. Since two peaks are identified in the heart rate histogram 700, an assumption can be made that only one of the two peaks provides an accurate heart rate estimate for the user, such as when the second peak is associated with artificial doubling resultant from secondary CGO peaks. In such cases, the lowest cluster (e.g., first peak 708) may be selected to be used for the calculated heart rate. The lowest cluster can be the cluster associated with the lowest heart rate. Selecting a cluster can include identifying a heart rate associated with the cluster (e.g., a heart rate associated with the peak maximum, a heart rate associated with the center of the peak, or the like), ignoring heart rate estimates not associated with the cluster and/or associated with another cluster, and/or applying different weighting values to heart rate estimates associated with the cluster and those not associated with the cluster.

In the example depicted in FIG. 7, point 716 is the median heart rate estimate from the flow data and point 714 is the median heart rate estimate acquired for the same individual via ECG data. While point 716 is somewhat close to the reference (point 714), the heart rate associated with the first peak 708 is even closer to the reference.

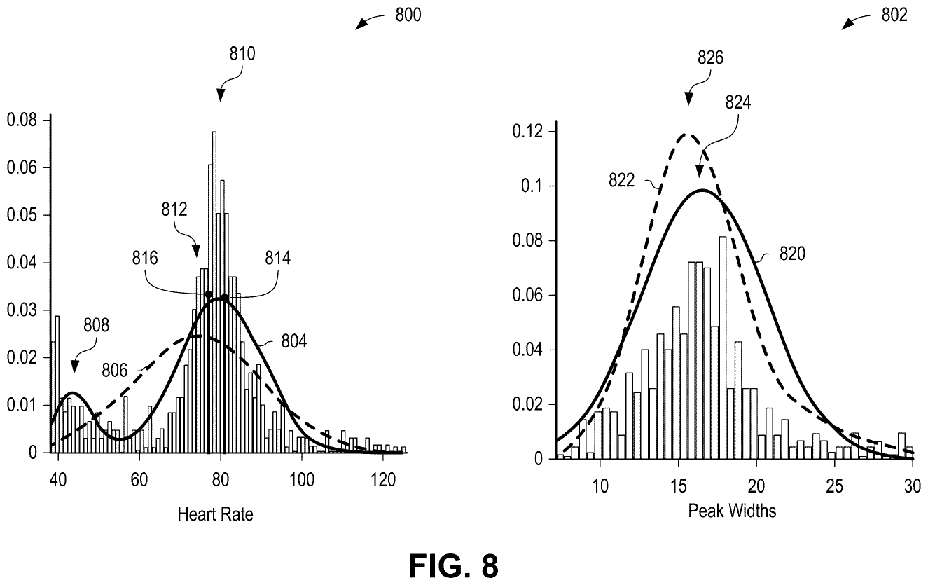

FIG. 8 is a set of histograms 800, 802 depicting the frequency of heart rate and peak width across a flow rate signal during an example sleep session, where the cardiogenic oscillations follow a single cluster of peak feature values, according to certain aspects of the present disclosure. The flow rate signal can be a flow rate signal similar to flow rate signal 502 of FIG. 5 or flow rate signal 602 of FIG. 6.

The heart rate histogram 800 and the peak morphology histogram 802 can be generated and processed similarly to as described above for respective histograms 700, 702 of FIG. 7.

As depicted in FIG. 8, for the peak feature histogram 802, curves 822, 820 are depicted. Curve 822 is generated based on a modeling approach configured to seek a single peak, whereas curve 820 is generated based on a modeling approach configured to seek two peaks. Other numbers of peaks can be used. Curve 822, as expected, shows a single peak 826. However, due to the nature of the peak widths identified for the sleep session used to generate the peak feature histogram 802, curve 820 also shows only a single peak 824, or at least any second peak is relatively negligible. Thus, a determination can be made that a single cluster of peak feature values exists in the identified CGO peaks.

In the heart rate histogram 800, a curve 806 seeking a single peak and/or a curve 804 seeking multiple peaks can be generated. The curve 806 shows a single peak 812 at approximately 75 beats per minute. The curve 804 shows a first peak 808 at approximately 43 beats per minute and a second peak 810 at approximately 80 beats per minute.

In some cases, based on the determination that a single cluster of peak feature values was identified (e.g., from peak feature histogram 802), a choice can be made to select the heart rate cluster (e.g., the peak) having the highest weighting (e.g., highest frequency of occurrence and/or largest area under the curve). In the example of histogram 800, second peak 810 has a higher weighting than first peak 808 (and peak 812), and thus can be selected to determine the heart rate information. Unlike the example of FIG. 7 where heart rate estimate cluster associate with the lowest heart rate is used, here the heart rate estimate cluster associated with the more common heart rate is used.

In the example depicted in FIG. 8, point 816 is the median heart rate estimate from the flow data and point 814 is the median heart rate estimate acquired for the same individual via ECG data. While point 816 is somewhat close to the reference (point 814), the heart rate associated with the second peak 810 is even closer to the reference.

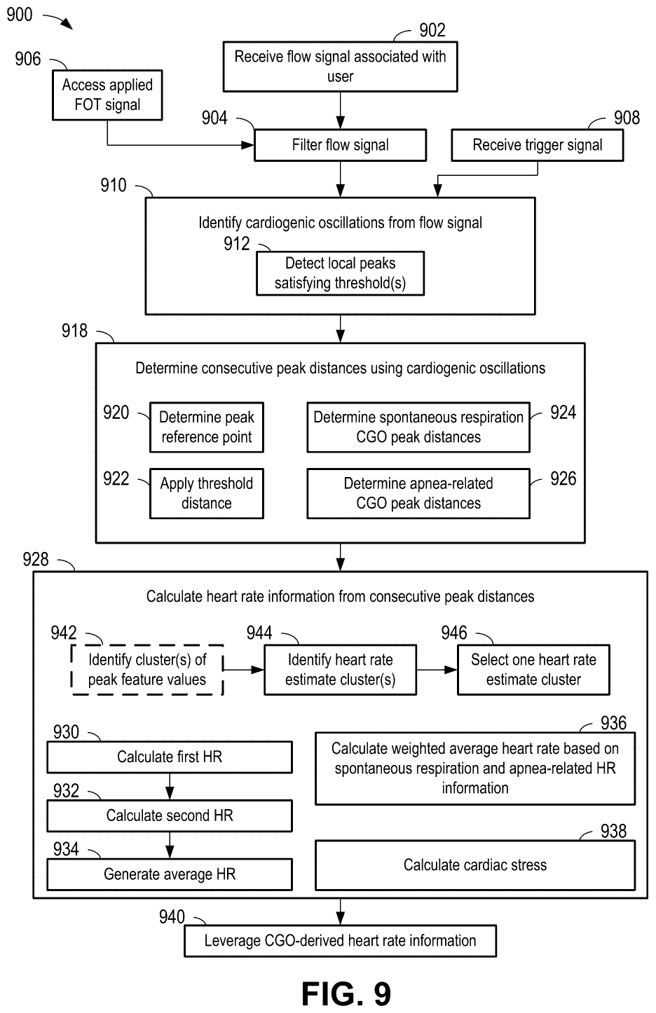

FIG. 9 is a flowchart depicting a process 900 for identifying cardiogenic oscillations and determining heart rate information according to certain aspects of the present disclosure. Process 900 can be performed by any suitable control system (e.g., control system 200 of FIG. 1).

At block 902, a flow signal associated with a user is received. The flow signal can be a flow rate signal or a flow pressure signal. The flow signal can be received from one or more sensors associated with airflow from the respiratory therapy device, through the conduit and/or the user interface to the user's airways, such as one or more flow rate sensors and/or one or more pressure sensors placed in or on a user interface, a conduit, or a respiratory therapy device. A flow rate signal can be flow rate data indicative of the rate of flow of air through the user interface over time. In some cases, a flow signal can be flow pressure data indicative of pressure of air (e.g., at the user interface, at the conduit, at the respiratory therapy device, or at the user's airway) over time.

At block 904, the flow signal can be filtered. Filtering the flow signal can include removing, from a flow signal, a FOT (forced oscillation technique) signal. The FOT signal is a forced oscillation of flow rate that is introduced by a respiratory therapy system for the purposes of diagnosing, or otherwise detecting or discrimination, a condition of the user. More specifically, FOT signals are often used to detect whether or not the user is experiencing a central apnea. The FOT signal can be accessed at block 906, such as being accessed from a respiratory therapy device applying the FOT. The FOT signal accessed at block 906 can be a signal representing FOT oscillations over time, or can simply be a frequency with which the FOT signal is applied. When the FOT signal is a frequency, removing the FOT signal can include passing the flow signal through a notch filter at that frequency. In some cases, the FOT signal is a pulse of pressure operating at a frequency of 4 Hz, in which case removing the FOT signal includes passing the flow signal through a notch filter that removes frequencies at or around 4 Hz. In some cases, the FOT signal is a signal indicating flow rate changes over time associated with only the FOT signal, in which case removing the FOT signal can include combining the flow signal with an inverse FOT signal. Without removing the FOT signal, the flow signal would include numerous artificial peaks due to the oscillating nature of the FOT signal, which would make identification of CGO peaks more difficult.

In some cases, filtering the flow signal at block 904 can include denoising the flow signal, such as by passing a flow signal through a low-pass filter or a smoothing filter. Removal of high-frequency noise (e.g., noise having frequencies at or above expected heart rate frequencies) can improve the identification of potential CGO peaks.

In some cases, filtering the flow signal at block 904 can include passing the flow signal through a filter to exaggerate localized peaks. More specifically, the flow signal can be passed through a Gaussian filter, which will exaggerate localized peaks, such as potential CGO peaks.

At block 910, cardiogenic oscillations (e.g., CGO peaks) can be identified from the flow signal (e.g., the filtered flow signal from block 904). Identifying CGOs can include applying a trained model or algorithm to the flow signal to identify whether or not a given localized peak is a CGO peak. Such a trained model or algorithm can take into account various features as disclosed in further detail herein.

In some cases, identifying CGOs can occur for the entire flow signal. In some cases, identifying CGOs can occur for only select portions of the flow signal based on a trigger signal. In such cases, the trigger signal can be used to only identify CGOs when heart rate information is needed and/or when the resultant heart rate information is likely to be sufficiently accurate. For example, in some cases, it may be desirable to obtain heart rate information from identified CGO peaks only during apnea events. The trigger signal can be based on any suitable signal. Examples of suitable trigger signals include an exhalation signal (e.g., triggers only during exhalations, such as detected from the flow signal); a sleep stage signal; a sleep state signal; an apnea event detection signal; and the like. In some cases, a sleep state signal (e.g., indication of whether or not the user is asleep) can be determined from the flow signal by analyzing the standard deviation of the flow signal over a window of time or by calculating breath volume stability over a window of time. Such trigger signals can be received at block 908.

In some cases, identifying CGOs at block 910 includes detecting local peaks that satisfy one or more peak feature thresholds at block 912. The peak feature thresholds can be those described with reference to FIG. 5, such as peak prominence, peak width, peak amplitude, peak distance, and the like. The thresholds (e.g., threshold values or threshold ranges) for these features can be trained as described herein.

In some cases, all localized peaks that satisfy the thresholds described with reference to block 912 can be identified as CGO peaks. In some cases, however, localized peaks that satisfy these thresholds can be identified as potential CGO peaks, which may be further identified as disclosed herein.

In some cases, identifying CGOs at block 910 can include detecting CGO peaks by performing Gaussian mixture modeling, or a similar cluster modeling approach, on peak features, or more specifically on values associated with peak features. Performing Gaussian mixture modeling on peak feature can include determining values for one or more peak features for a set of potential peaks, then applying Gaussian mixture modeling on these values to identify clusters of peak feature values. A cluster of peak feature values can be a localized peak based on the frequency that certain values appear for that feature across the potential peaks. For example, when using peak width as the feature, a set of potential peaks may have peak widths that span from 10 to 30 samples, with most of the potential peaks having peak widths at 13 samples and 25 samples. Thus, a histogram depicting these peaks would itself have localized peaks (e.g., clusters of peak feature values) around 13 and 25. Depending on the nature of the feature values, the number of clusters of peak feature values can be 1, 2, 3, or more. In some cases, Gaussian mixture modeling or other modeling approaches can be set up to seek 1, 2, 3, or more peaks.

Once the clusters of peak feature values have been identified, one of the clusters of peak feature values can be selected as the peak associated with CGOs (e.g., primary CGOs), and the peak features defined by that cluster (e.g., for the cluster at 13 samples in the example above, the defined peak feature would be a peak having a width of 13 samples or within a threshold, such as the standard deviation of the distribution that is associated with that cluster) 13 samples) can used to identify which of the potential peaks are CGO peaks and/or which of the CGO peaks are primary CGO peaks. In some cases, clusters of peak features can be based on multiple features (e.g., peak width and peak prominence). In some cases, potential peaks can be assigned a likelihood of being a CGO peak based on proximity to the feature values of the selected cluster of peak feature values (e.g., in the previous example, a peak with a width of 14 samples is more likely to be a CGO peak than a peak with a width of 19 samples).

In some cases, selecting the cluster of peak features can be based on determining estimated heart rates for each of the different clusters of peak feature values (e.g., in the example above, a first heart rate if the cluster associated with 13 samples is selected and a second heart rate if the cluster associated with 25 samples is selected), then selecting the cluster of peak feature values associated with the more likely accurate heart rate. In some cases, the more likely accurate heart rate is simply the lowest option heart rate.

In some cases, selecting the cluster of peak feature values to use can be done without substantial analysis, such as by picking a random cluster of peak feature values; picking the cluster of peak feature values based on its cardinal number (e.g., always picking the first peak); picking the cluster of peak feature values based on its amplitude, area-under-the-curve, width; or the like. This approach can be especially useful when the potential peaks include CGO peaks and doubled CGO peaks.

In some cases, features other than peak features can be used to identify CGOs. Such features can also be trained using training similar to how peak features are trained as described with reference to FIG. 5. For example, breathing stability can be determined by isolating peaks that occur when the user is likely asleep, such as using a standard deviation of the flow signal over a window of time or calculating breath volume stability over a window of time. Peaks meeting a threshold breathing stability score may be classified as CGO peaks. In another example, breath phase could be used to facilitate CGO identification, which can include identifying peaks as CGO peaks based on a time since last exhale. In some cases, an exhale ratio value can be a feature used to facilitate CGO identification, in which the exhale ratio value is a ratio between a current flow rate (or flow pressure) and the flow rate (or flow pressure) at peak exhale. In some cases, a measure of entropy can be used as a feature to facilitate CGO identification, in which case statistical entropy can be calculated over a window of time (e.g., the past breath cycle, the last 10 seconds, the last epoch, etc.) and used to identify CGO peaks.

At block 918, peak distances can be calculated between consecutive identified CGOs (e.g., peaks identified as CGO peaks and/or primary CGO peaks) from block 910. Calculating peak distances at block 918 can include identifying a peak reference point at block 920, which can be a point of reference from which distances along the time axis are calculated. Examples of peak reference points include a peak center (e.g., a time-based center or a volume-based center), a peak apex, or the like.

In some cases, a peak center can refer to a time-based center, which can be a point that is halfway between a starting point and an ending point of the peak. In some cases, a peak center can refer to a volume-based center, which can be a point which evenly splits the area-under-the-curve of the peak.

In some cases, the CGO peak center falls between two samples. To find out whether the current CGO peak location is slightly before or after the identified sample index, the ratio between the current, previous and following flow values is calculated. This offset, C2, is calculated as follows:

$${C}_{2} = \frac{\vert X ( {m}_{k + 1} ) \vert - \vert X ( {m}_{k - 1} ) \vert}{4 \vert X ( {m}_{k} ) \vert - 2 \vert X ( {m}_{k - 1} ) \vert - 2 \vert X ( {m}_{k + 1} ) \vert}$$

Where mₖ is the sample index for the identified CGO peak, mₖ₋₁ is the previous sample index, mₖ₊₁ is the following sample index, X is the flow signal associated with the flow data and C₂ is the offset that is applied to the current CGO peak index.

The current CGO peak location, mₖ, can now be updated with this fractional offset, according to the following equation:

$${m}_{k} = {m}_{k} + {C}_{2}$$

When mₖ is updated thusly, mₖ may no longer have an integer value.

In an example, a CGO peak may fall somewhere between the 500^{th} sample and the 501^{st} sample. Instead of using a value of 500 or 501 to represent the CGO peak's center, C₂ may be calculated as shown above and added to 500 to obtain a new value to be used as the CGO's peak center. For example, if C₂ is 0.48, then the new value for the CGO peak's center may be 500.48.

In some cases, determining the peak center for a given cardiogenic oscillation includes using i) autocorrelation; ii) wavelet transforms; or iii) signal decomposition. Autocorrelation can involve taking a segment of a signal and sliding one copy of it over another, with one copy remaining stable at time 0 and the other moving to the right at each time increment. At each increment of time, t, the correlation of the stationary signal and the moving signal is calculated. The output returns how correlated the signals are at each time point, and for a sinusoidal signal, the correlation output would be sinusoidal as such a signal correlates most when it aligns well with the next sinusoid. In the case of CGO peaks, a segment of the stable portion of the exhalation period of a breath can be used as a sample that can be slid across itself to determine correlation. If there is a repeating pattern within the exhalation period, the autocorrelation signal will show peaks at where the repeats occur. A peak detection algorithm can then be applied to decipher thresholds at which the correlations correspond to CGOs. Autocorrelation may be especially effective in cases of a central apnea where the flow signal is initially stable.

Wavelet transforms involves selecting a mother wavelet shape that can exploit the shape of the signal being identified. The mother wavelet can be convolved with the flow signal for all times t, as well as being stretched to convolve with the flow signal at a range of frequencies. The width of the wavelet corresponds to the frequency. The advantage of a wavelet transform is that multiple wavelets could be used, such as to pick up cases where duplicated CGO peaks occur. In some cases, wavelet transforms are especially effective in cases of central apneas. Like autocorrelation, a peak detection algorithm can be used.

Signal decomposition can involve breaking a signal down into a selection of frequencies that make up the signal. By separating the signal into different components, components that likely relate to CGO frequencies can be isolated.

In some cases, determining consecutive peak distances at block 918 can include applying a threshold distance at block 922 to determine whether a first CGO peak and a next CGO peak should be treated as consecutive peaks. The threshold distance can be a value that is the minimum or maximum acceptable distance between two peaks to count them as consecutive peaks. For example, if two peaks are separated by too large of a distance, an assumption can be made that at least one heart beat occurred between the two peaks, and thus the two peaks should not be treated as consecutive peaks. In some cases, if the distance between two peaks is so short that an instantaneous heart rate calculated from the two peaks would be unlikely or impossible, those two peaks should not be treated as consecutive peaks. In some cases, the threshold distance applied at block 922 is similar to peak distance 516 of FIG. 5. Acceptable tolerances for threshold distances may be pre-defined such as the a maximum and/or minimum distance one may expect for a valid heart rate (e.g., if it is desired to consider anything <35 bpm as invalid, the maximum threshold distance can be set at a distance that would equate to 35 bmp, or likewise, if it is desired to consider anything >180 bpm as invalid, the minimum threshold distance can be set at a distance that would equate to 180 bpm).

In some cases, at block 918, determining consecutive peak distances include determining a set of CGO peak distances associated with spontaneous respiration at block 924 and/or determining a set of CGO peak distances associated with apnea events at block 926. In such cases, heart rates determined from the spontaneous respiration CGO peak distances can be heart rates associated with spontaneous respiration (e.g., heart rates that occur while the user is breathing regularly), and heart rates determined from the apnea-related CGO peak distances can be heart rates associated with apnea events (e.g., heart rates that occur while the user is experiencing apnea events). An apnea event signal can be used to differentiate CGO peaks associated with apnea events from those associated with spontaneous respiration.

At block 928, the peak distance(s) from block 918 can be used to calculate heart rate information. Calculating heart rate information can include calculating a heart rate, heart rate variability, cardiac stress, or other heart-rate-related information. Calculating heart rate information can include calculating a number of heart rate estimates based on the consecutive peak distances from block 918. Each of the heart rate estimates can represent an instantaneous heart rate value.

In some cases, all heart rate estimates may be accurate or sufficiently accurate, such as in cases where all CGO peaks are properly identified, no false peaks are identified as CGO peaks, and only primary CGO peaks exist (e.g., no secondary CGO peaks). However, that may not always be the case. In some cases, artifacts, secondary CGO peaks, and other issues may cause some of the heart rate estimates to be inaccurate. For example, secondary CGO peaks may result in heart rate estimates that are greater than (e.g., twice the value of) the user's actual heart rate. In some cases, calculating heart rate information can include determining heart rate information from a portion of the heart rate estimates, excluding or ignoring a portion of the heart rate estimates, and/or applying weighting values to different portions of the heart rate estimates (e.g., weighing heart rate estimates presumed to be accurate more highly than those presumed to be inaccurate).

In some cases, calculating heart rate information at block 928 can include identifying one or more heart rate estimate clusters at block 944, such as described with reference to FIGS. 7-8. Identifying a heart rate estimate cluster can include determining one or more peaks in a curve representing the frequency of heart rate estimates for a set of heart rate values (e.g., identifying clusters of heart rate value(s) found in the batch of heart rate estimates). Each peak can define a respective heart rate estimate cluster. In some cases, the curve can be generated using an appropriate modeling approach, such as Gaussian mixture modeling, which can be configured to seek one, two, three, or more peaks.

Once the heart rate estimate clusters have been identified at block 944, one of the heart rate estimate clusters can be selected at block 946. In some cases, selecting a heart rate cluster can include simply selecting the only heart rate cluster that is identified. In some cases, selecting a heart rate cluster can include selecting the lowest heart rate cluster (e.g., the heart rate cluster associated with the lowest heart rate(s) out of all the heart rate clusters) or selecting the heart rate cluster with the highest associated frequency of heart rate estimates (e.g., the heart rate cluster having the highest curve peak and/or highest area under the curve in the histogram of heart rate values).

In some cases, selecting a heart rate estimate cluster at block 946 can be dependent on a number of clusters of peak feature values present in the identified CGO peaks. In some cases, the number of clusters of peak feature values can determined at block 942. At block 942, one or more clusters of peak feature values can be identified and the number of clusters of peak feature values can be used in the selection of one of the heart rate estimate clusters at block 946.

Identifying one or more clusters of peak feature values at block 942 can include identifying one or more clusters of peak feature values associated with one or more peak features such as described with reference to FIGS. 7-8. Identifying a cluster of peak feature values can include determining one or more peaks in a curve representing the frequency of values for a set of one or more peak feature values (e.g., identifying clusters of peak feature values found in a batch of peak features). Each peak can define a respective cluster of peak feature values. In some cases, the curve can be generated using an appropriate modeling approach, such as Gaussian mixture modeling, which can be configured to seek one, two, three, or more peaks. The peak feature values can be values associated with one or more peak features, such as those peak features described with reference to FIG. 5. In some cases, the peak feature used at block 942 is peak width.

Once a heart rate estimate cluster has been selected at block 946, it can be used to determine heart rate data, such as a heart rate, heart rate variability, and other heart rate information. The heart rate data can be estimated by making use of a portion of the heart rate estimates associated with the heart rate estimate cluster (e.g., the heart rate estimates within a threshold of the peak associated with the heart rate estimate cluster, such as within a standard deviation thereof), by making use of data associated with the peak associated with the heart rate estimate cluster (e.g., a location of the peak, a location of an apex of the peak, a location of a center or centroid of the peak), or otherwise leveraging the selected heart rate estimate cluster.

In some cases, calculating heart rate information at block 928 can include calculating a first heart rate (e.g., a first instantaneous heart rate) at block 930 from a first consecutive peak distance associated with a first set of CGO peaks, and calculating a second heart rate (e.g., a second instantaneous heart rate) at block 930 from a second consecutive peak distance associated with a second set of CGO peaks. The first set of CGO peaks and second set of CGO peaks can overlap or can be entirely separate, separated by any amount of time. At block 934, an average heart rate can be calculated based on the first heart rate from block 930 and the second heart rate from block 932. In some cases, the average heart rate can be further calculated based on additional heart rates, such as all heart rate estimates collected for a given sleep session.

In some cases, at block 936, calculating heart rate information at block 926 includes determining heart rate information associated with spontaneous respiration (e.g., from the spontaneous respiration CGO peak distances of block 924), determining heart rate information associated with apnea events (e.g., from the apnea-related CGO peak distances of block 926), and applying weighting values to each to achieve a weighted average heart rate. This weighted average heart rate can be useful to emphasize CGO-derived heart rates from apnea events over those from spontaneous respiration, or vice versa. In some cases, CGO-derived heart rates may be more accurate during apnea events, in which case apnea-related heart rate information may be more highly weighted than spontaneous-respiration-related heart rate information.

In some cases, calculating heart rate information at block 926 includes calculating cardiac stress at block 938. Cardiac stress can be calculated based on a change in heart rate between two distinguishable times or conditions. In an example, stress due to apnea can be a useful metric to identify and visualize the impact apnea has on an individual. Stress due to apnea can be calculated by determining a heart rate during an apnea event (e.g., an apnea-related heart rate) and a heart rate not during an apnea event (e.g., spontaneous-respiration-related heart rate), then determining a change in heart rate between the apnea-related heart rate and the non-apnea-related heart rate. This change in heart rate can be used to estimate an amount of stress imposed on the user during an apnea event following an apnea event. This level of stress can be presented to the user to help the user understand how their body reacts to apneas, and to urge the user to remain compliant with any sleep therapies that could reduce apneas.

In some cases, heart rate information can be used to identify abnormalities in heart rate, such as arrhythmias. Heart rate information can also be used to identify stress associated with a sleep session, such as in cases where heart rate over a large portion of the sleep session increases, rather than decreases, as expected for restful sleep. It is expected that in restful sleep, heart rate would decrease during sleep and start increasing again soon before the patient wakes up.

In some cases, calculating heart rate information at block 928 can include generating a confidence value associated with the heart rate information. The confidence value can be indicative of the degree of confidence that the CGO-derived heart rate information is consistent with ECG-derived heart rate information. The confidence value can be based on one or more variables. In some cases, determining such a confidence value can be based on a calculation of CGO uptime. It has been found that CGO-derived heart rate information can be more accurate in instances with higher CGO uptime.

In an example, as an alternative to or along with a confidence value, an invalid warning can be issued to indicate when particular CGO-derived heart rate information may be invalid. Such a warning can be issued based on one or more conditions, such as the CGO uptime falling below a threshold value and heart rate distribution peaks being broad (e.g., having a large standard deviation). In an example, heart rate information may be identified as invalid when the overall CGO uptime is below a threshold value and when a frequency of the identified cardiogenic oscillations that fall within a threshold distance of at least one of the set of heart rate distribution peaks is below a threshold frequency value.

In some case, calculating the heart rate information at block 928 can also include calculating a stability metric. The stability metric is an indication of the stability of the calculated heart rate information over time. In some cases, if the stability metric is indicative of unstable heart rate information over time (e.g., large variations between adjacent readings), an invalidation warning may be issued to indicate that the heart rate information may not be valid. In some cases, the stability metric can be used to identify arrhythmias or other heart conditions that may generate an unstable heart rate.

At block 940, the CGO-derived heart rate information from block 928 can be leveraged in various ways. In some cases, leveraging CGO-derived heart rate information can include monitoring cardiac conditions and overall cardiac health. For example, higher resting heart rates can be associated with increased mortality risk. Since users of respiratory therapy devices generally use their devices while sleeping and over the course of many days, weeks, months, or years, CGO-derived heart rate information can be a very convenient and unobtrusive technique for obtaining resting heart rate data and for identifying changes in resting heart rate over large timescales (e.g., the order of days, weeks, months, years, or more). CGO-derived heart rate information can be leveraged to show the efficacy of respiratory therapy, as continued use of respiratory therapy may improve metrics such as resting heart rate. Other metrics and information associated with CGO-derived heart rate information can be shown to the user or used to generate visualizations for the user to help the user better understand their health, better understand the benefit they obtain from respiratory therapy, and otherwise improve therapy adherence. In an example, if a user stops engaging in respiratory therapy for a number of sleep sessions, then decides to start up again, CGO-derived heart rate information from the most recent sleep session(s) can be compared with CGO-derived heart rate information from before the user's break in respiratory therapy, which may show that the user's heart rate is now higher and thus indicative of more cardiac stress (which can be termed a “rebound effect”), which may show the user how they benefited from respiratory therapy.

In some cases, leveraging CGO-derived heart rate information at block 940 includes using the heart rate data to provide augmented sleep-related analytics. By leveraging heart rate information during use of a respiratory therapy device along with other sleep-related data, various sleep-related models and metrics can be improved.

In some cases, leveraging CGO-derived heart rate information at block 940 includes reporting the CGO-derived heart rate information to healthcare providers. Such reporting can help healthcare providers provide tailored care to the user. In an example, the heart rate information can be used to ensure new users are receiving beneficial therapy. In another example, the heart rate information can be used to help healthcare providers identify users with potential cardiac risks by identifying a change in cardiac behavior with respect to the patient's baseline behavior.

In some cases, some or all of process 900 can occur primarily in real-time, calculating heart rate information as the user is making use of the respiratory therapy device. In some cases, however, some or all of process 900 can occur after completion of a sleep session.

While process 900 is depicted with certain blocks in a certain order, in some cases, process 900 can be performed using fewer blocks, more blocks, or different blocks than those shown. Additionally, in some cases, certain blocks can be performed in different orders and/or certain sub-blocks can be adapted to operate in different parent blocks. For example, in some cases, instead of determining spontaneous respiration CGO peak distances at block 924 and apnea-related CGO peak distances at block 926, a version of process 900 can simply calculate CGO peak distances at block 918 and calculating heart rate information at block 928 can include differentiating the CGO peak distances into those related to spontaneous respiration and those related to apnea events, then performing actions on those differentiated CGO peak distances.

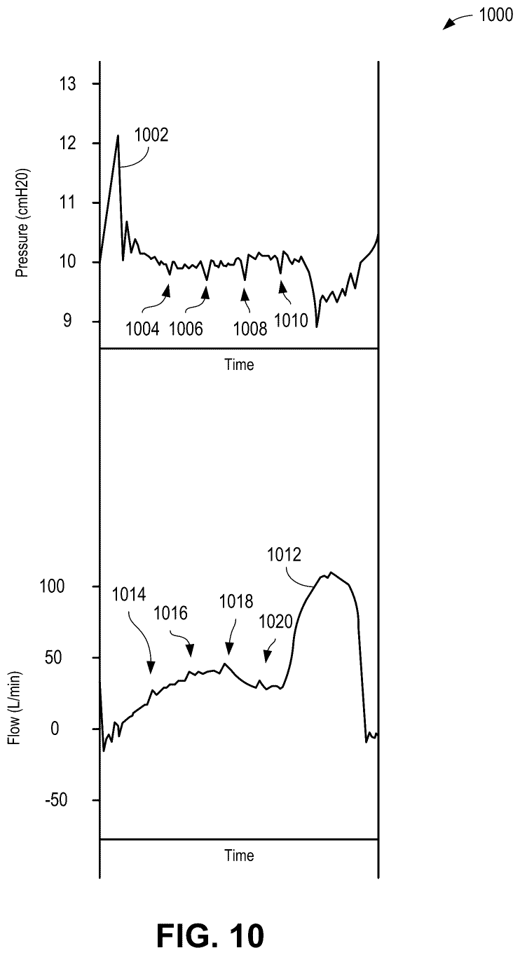

FIG. 10 is a combination chart 1000 depicting cardiogenic oscillations present in pressure and flow signals over time, according to certain aspects of the present disclosure. The flow rate is shown by a flow rate signal 1012, which traces the flow rate over time of pressurized air from a respiratory therapy device through a conduit and/or user interface (e.g., user interface 120 of FIG. 1) to a user's airways. The air pressure is shown by flow pressure signal 1002, which traces the air pressure over time of the pressurized air. The air pressure data can come from any suitable sensor, such as a pressure sensor in a user interface, in a conduit, in a respiratory therapy device, or the like. The flow rate signal 1012 and pressure signal 1002 can be measured by one or more sensors (e.g., flow rate sensor(s) 214 and pressure sensor(s) 212 of FIG. 1).

CGO peaks 1004, 1006, 1008, 1010 are present in the pressure signal 1002, as shown by localized peaks in the pressure signal 1002. As seen in FIG. 10, the localized peaks take the form of dips (e.g., with the tip of the peak reaching below the baseline of the pressure signal 1002). CGO peaks 1014, 1016, 1018, 1020 are present in the flow rate signal 1012, as shown by localized peaks in the flow rate signal 1012. For the example depicted in FIG. 10, the CGO peaks 1004, 1006, 1008, 1010 from the pressure signal 1002 align with the CGO peaks 1014, 1016, 1018, 1020 in the flow rate signal 1012.

One or more elements or aspects or steps, or any portion(s) thereof, from one or more of any of claims 1 to 70 below can be combined with one or more elements or aspects or steps, or any portion(s) thereof, from one or more of any of the other claims 1 to 70 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving a flow signal associated with air supplied to airways of a user engaging in a sleep session, the air being supplied by a respiratory therapy device;
identifying a plurality of cardiogenic oscillations from the flow signal;
determining consecutive peak distances based at least in part on the identified plurality of cardiogenic oscillations; and
calculating heart rate information based on the consecutive peak distances.

2-37. (canceled)

38. A system comprising:
a flow generator of a respiratory therapy device for supplying pressurized air to airways of a user engaging in a sleep session;
one or more flow sensors for supplying a flow signal associated with the supplied pressurized air;
a control system comprising one or more processors; and
a non-transitory computer readable medium having thereon machine executable instruction, which, when executed by the one or more processors, cause the control system to perform operations including:
receiving the flow signal;
identifying a plurality of cardiogenic oscillations from the flow signal;
determining consecutive peak distances based at least in part on the identified plurality of cardiogenic oscillations; and
calculating heart rate information based on the consecutive peak distances.

39-70. (canceled)
